(19) United States
(12) Patent Application Publication
(10) Pub. No.: US 2026/0079146 A1
(43) Pub. Date: Mar. 19, 2026

(54) RECREATIONAL WATER MANAGEMENT USING OXIDATION-REDUCTION POTENTIAL DATA

(71) Applicant: King Technology, Inc., Minnetonka, MN (US)

(72) Inventors: Daniil A. Rezekulov, Savage, MN (US); Daniel P. Longhenry, Bloomington, MN (US)

(73) Assignee: King Technology, Inc., Minnetonka, MN (US)

(21) Appl. No.: 19/325,136

(22) Filed: Sep. 10, 2025

Related U.S. Application Data
(60) Provisional application No. 63/694,419, filed on Sep. 13, 2024.

(51) Int. Cl. G01N 33/18; C02F 1/467; C02F 1/68; C02F 103/42; G01N 27/416

(52) U.S. Cl. CPC G01N 33/18; C02F 1/4674; C02F 1/68; G01N 27/4168; C02F 2103/42; C02F 2209/04

(57) ABSTRACT

The invention provides various methods of managing a recreational body of water. In some embodiments, the invention provides methods for assessing whether a recreational body of water that previously contained 5,5-dimethylhydantoin (DMH) still contains DMH. Furthermore, some embodiments provide a method of testing a recreational body of water that includes DMH by taking a series of oxidation-reduction potential (ORP) measurements for the water while it has a free chlorine level in a range of about 0.5 to 1.0 ppm, and in response to those measurements, determining whether the DMH present in the water is undergoing decomposition.

### RELATED APPLICATIONS

This application claims priority to U.S. Provisional Patent Application No. 63/694,419, filed Sep. 13, 2024, the entire contents which are incorporated herein by reference.

### TECHNICAL FIELD

This disclosure relates generally to methods of managing recreational bodies of water. More specifically, this disclosure relates to assessing whether a recreational body of water has sufficient chlorine or, in contrast, has insufficient chlorine and is likely contaminated.

### BACKGROUND

The water chemistry in recreational bodies of water (e.g., pools, hot tubs, and spas) should be managed to provide safe, comfortable water for bathers. In many cases, it is desirable to monitor chlorine levels in such bodies of water. The most accurate way to measure chlorine directly may be to use chemistry kits. These kits, however, are more complicated than would be ideal for the average consumer. Single-use test strips are an easier option, but they may have suboptimal resolution, and thus may be subjective or even misleading. It would be desirable to provide a simple, yet more reliable and/or more objective, way to assess chlorine levels in recreational bodies of water.

Oxygen reduction potential (“ORP”) is a measurement used to assess the ability of chlorine in a recreational body of water to oxidize contaminants in the water. For a hot tub or spa filled with water of conventional chemistry and running 0.5-1 ppm free chlorine in a proper pH range, a typical ORP signature is roughly 700-750 mV. Thus, according to conventional ORP logic, recreational water with an ORP level well below 700-750 mV would be expected to be contaminated.

### SUMMARY

In some embodiments, the invention provides a method for assessing whether a recreational body of water that previously contained 5,5-dimethylhydantoin (DMH) still contains DMH. In the present embodiments, the method includes taking a series of oxidation-reduction potential (ORP) measurements for the water while it has a free chlorine level in a range of about 0.5 to 1.0 ppm. In addition, the present method includes determining DMH to be present in the water in response to at least a majority of the ORP measurements being in a range of between 100 and 600 mV.

Certain embodiments of the invention provide a method for assessing whether a recreational body of water that previously contained 5,5-dimethylhydantoin (DMH) still contains DMH. In the present embodiments, the method includes taking an oxidation-reduction potential (ORP) measurement for the water while it has a free chlorine level in a range of about 0.5 to 1.0 ppm. In addition, the present method includes determining DMH to be present in the water in response to the ORP measurement being below 500 mV.

Some embodiments of the invention provide a method for assessing whether a recreational body of water that previously contained 5,5-dimethylhydantoin (DMH) still contains DMH. In the present embodiments, the method includes taking an oxidation-reduction potential (ORP) measurement for the water while it has a free chlorine level of at least 0.5 ppm. In addition, the present method includes determining DMH to be absent from the water in response to the ORP measurement being greater than 700 mV.

In certain embodiments, the invention provides a method of testing a recreational body of water that includes 5,5-dimethylhydantoin (DMH). In the present embodiments, the method includes taking a series of oxidation-reduction potential (ORP) measurements for the water while it has a free chlorine level in a range of about 0.5 to 1.0 ppm. In addition, the present method includes, in response to the ORP measurements reflecting a gradual decrease of ORP level in the water, determining that the DMH present in the water is undergoing decomposition.

While multiple embodiments are disclosed herein, still other embodiments of the present disclosure will become apparent to those skilled in the art from the following detailed description, which describes illustrative embodiments of the disclosure. Accordingly, the detailed description is to be regarded as illustrative in nature and not restrictive.

The details of one example are set forth in the accompanying drawing and the related description section. Other features, objects, and advantages will be apparent to skilled artisans given the present description, drawing, and claims.

### BRIEF DESCRIPTION OF DRAWING

FIG. 1 is a graph of ORP measurements taken for water in a hot tub over a period of about 61 days.

FIG. 2 is a graph of ORP measurements taken for water in another hot tub over a period of about 37 days.

FIG. 3 is a graph of other ORP measurements taken for water in the hot tub of FIG. 2 over a period of about 18 days.

### DETAILED DESCRIPTION

The following detailed description is to be read with reference to the drawing. The drawing exemplifies certain preferred embodiments and is not intended to limit the scope of the invention. Skilled artisans will recognize that the examples and embodiments described herein have many useful alternatives that fall within the scope of the invention.

So that the present disclosure may be more readily understood, certain terms may be defined. Unless otherwise defined, all technical and scientific terms used herein have the meaning commonly understood by one of ordinary skill in the art to which the technology of this disclosure pertains.

Numeric ranges recited in the specification are to be understood to disclose and recite all the numbers defining the range and each integer within the defined range. Throughout this disclosure, various aspects are presented in range format. The descriptions in range format are merely for example, convenience, and/or brevity and should not be construed as an inflexible limitation on the scope of the disclosure. Moreover, the description of a range is to be understood as also specifically disclosing all possible sub-ranges, fractions, and individual numerical values within that range. For example, description of a range from 1 to 6 is to be considered as also specifically disclosing sub-ranges like from 1 to 3, from 1 to 4, from 1 to 5, from 2 to 4, from 2 to 6, from 3 to 6, etc., as well as each individual number within that range, i.e., 1, 2, 3, 4, 5, and 6, and relevant decimals and fractions, for example, 1.2, 3.8, 1½, and 4¾. This applies regardless of the breadth of the range.

Furthermore, in the present disclosure, anywhere the terms “comprising” or “comprises” are used, those terms have their ordinary, open-ended meaning. In addition, where appropriate, the disclosure at each such location is to be understood to also disclose that it may, as an alternative, “consist essentially of” or “consist of.”

A recreational body of water is understood to comprise a body of water (e.g., a pool, spa, hot tub, or the like) in which a person wholly or partially immerses themself. In any embodiment of the present disclosure, the recreational body of water can optionally be a pool, hot tub, or spa, including a swim spa. While the methods described herein are suitable for managing various recreational bodies of water, certain embodiments involve a pool, such as an outdoor pool (e.g., a seasonal outdoor pool). Any method of the present disclosure can optionally be performed with a pool, such as an outdoor pool. The chlorine needs of an outdoor pool can vary for different reasons, including bather load and environmental loads.

As is well known to those of ordinary skill in this field, free chlorine refers to the amount of hypochlorous acid (HOCl) and hypochlorite (ClO) in a body of water that have not yet reacted with contaminants.

As is also well known to those of ordinary skill in this field, total chlorine refers to the total amount of free chlorine plus combined chlorine. Combined chlorine generally refers to chlorine that is bound to contaminants (e.g., chloramines).

In certain embodiments, the water of the recreational body of water comprises (e.g., is) salt water. Thus, the present methods can optionally include adding sodium chloride, such as by adding pool salt, to the recreational body of water. In such cases, the method may further include, during an initial stage that includes providing fresh water, adding chlorine in another form, such as by adding bleach or calcium hypochlorite (optionally bringing total chlorine level to a range of about 1-20 ppm, perhaps 3-5 ppm). In some embodiments involving salt water, an electrolytic chlorine generator is coupled operatively with the recreational body of water. Furthermore, the water can optionally be salt water in any embodiment of this disclosure. Thus, the present methods may be performed with a pool, hot tub, or spa filled with salt water. It is to be appreciated, however, that the embodiments and methods of this disclosure are by no means limited to salt water.

For a hot tub or spa filled with water of conventional chemistry (e.g., not containing dimethylhydantoin (“DMH”)) and running at about 0.5-1 ppm free chlorine in a proper pH range (e.g., about 7.2-7.8), a typical ORP signature is roughly 700-750 mV. Such a high level of ORP is shown schematically by the horizontal dashed line at the top region of Graph 1 below.

According to conventional ORP logic, recreational water with an ORP level that is well below 700-750 mV would be expected to be contaminated. Applicant has discovered, however, that when the recreational body of water includes DMH, the water can surprisingly be in good condition for bather use (e.g., can have free chlorine in a desired range of about 0.5-1 ppm) at lower ORP levels, such as less than 700 mV, less than 650 mV, less than 600 mV (e.g., about 300-500 mV), or even less than 500 mV.

In the schematic graph above, the lower dashed line schematically characterizes behavior of a recreational body of water that includes DMH and is running at about 0.5-1 ppm free chlorine in a proper pH range (e.g., about 7.2-7.8). The illustrated line has an initial extent that is horizontal, which reflects a relatively constant ORP signature. This initial stage, when it occurs, tends to last longer when a larger initial amount of DMH is used. By comparison, in cases where a smaller initial amount of DMH is used, such an initial stage may be of shorter duration. There may even be no such discernable initial stage. In cases where this type of initial stage occurs, it may be characterized by ORP signature in a range of, for example, 300-650 mV, such as about 300-600 mV. During this type of initial stage, the free chlorine level tends to stay relatively constant (e.g., in a desired range of about 0.5-1 ppm). In some cases, ORP measurements taken during such an initial stage may encompass and/or be generally centered around (e.g., with some above, and some below) 500 mV. This, however, is by no means always the case. Rather, the ORP levels for different systems will vary based on multiple factors, such as the size of the recreational body of water, the amount of DMH added to the water, and water chemistry.

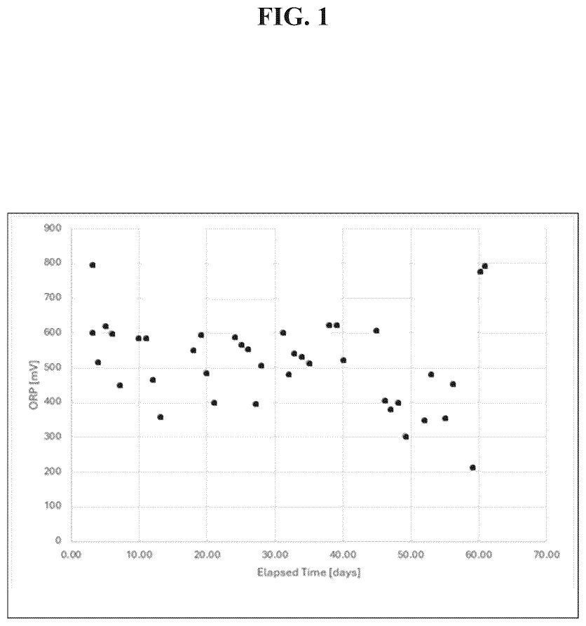

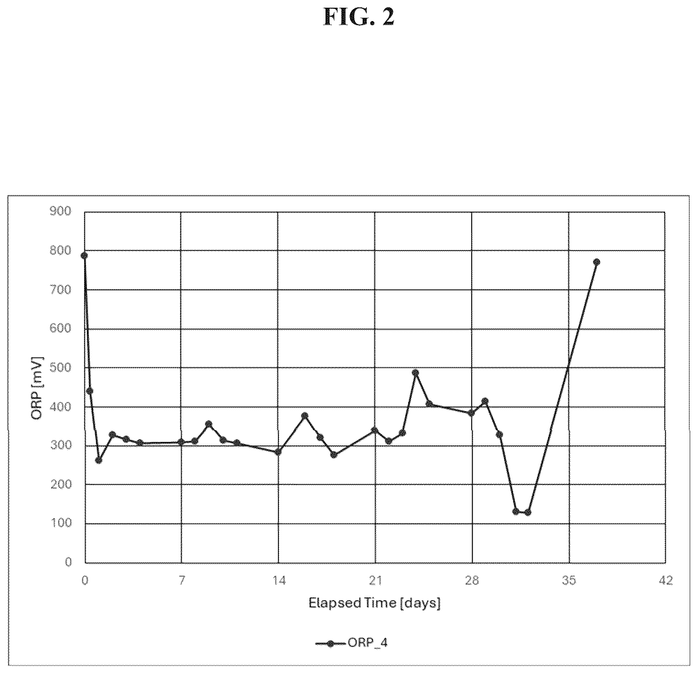

In some cases where this type of initial stage occurs, it may last more than 10 days, more than 15 days, or even more than 20 days. In certain embodiments of this nature, such an initial stage is preceded by adding DMH to the body of water to establish therein at least 35 ppm DMH, at least 40 ppm DMH, at least 45 ppm DMH, or more. In the example of FIG. 1, the initial stage is shown spanning from about day 4 to about days 40-45. In the example of FIG. 2, the initial stage is shown spanning from about day 2 to about day 28. It is to be appreciated, however, that this will depend upon various factors, such as bather load, water volume, initial amount of DMH, and water chemistry. As noted above, for example, smaller initial amounts of DMH will tend to provide shorter initial stages of this nature, or even no such discernable initial stage.

As time passes, DMH in the water decomposes. Accordingly, the ORP measurements reflect a decrease of the DMH level in the water. This can be seen at the decline stage of Graph 1 above; the decline stage is encompassed by the dashed-line box. In the schematic illustration of Graph 1, the ORP signature is shown having a downward concave curve. While this may be the case in some examples, the gradual decline of ORP is by no means always characterized by a downward concave curve. During the decline stage, the free chlorine level tends to stay relatively constant (preferably in the desired range of about 0.5-1 ppm). The decline stage may last for about 1-3 weeks, such as from 5 to 20 days, or about 1-2 weeks, such as from 6 to 18 days. In some cases, the decline stage lasts at least five days, at least seven days, at least 10 days, at least 12 days, or at least 14 days. In certain examples, the decline stage lasts about 1-2 weeks. Applicant has discovered that the gradual decrease of ORP can be used to predict when the DMH in the water will be gone. More will be said of this later.

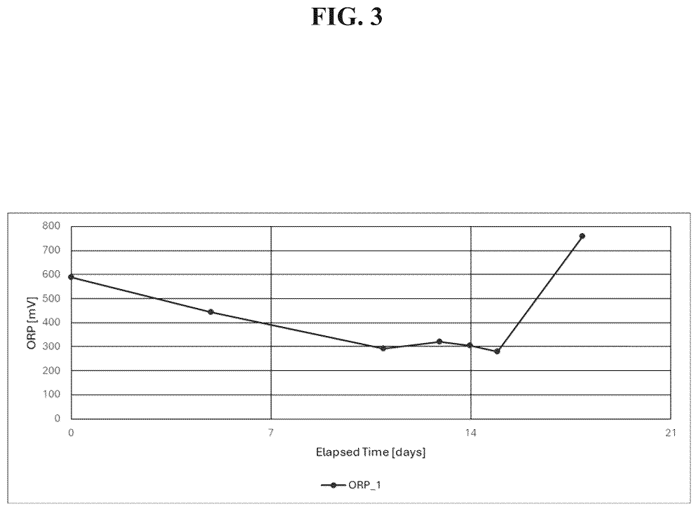

While Graph 1 above shows distinct initial and decline stages, this is not required. As noted above, in cases where a smaller initial amount of DMH is used, there may be no discernable initial stage. Instead, there may be a more continuous decrease, as shown in the non-limiting example of FIG. 3. Either way, the gradual decline of ORP can be used advantageously in testing, assessing, and/or managing a body or recreational water. Also, the free chlorine level tends to remain relatively constant (e.g., between about 0.5-1 ppm) throughout an initial stage when ORP is staying relatively constant (if this occurs) and/or throughout a decline stage when ORP is declining in a general and/or gradual manner.

Thus, Applicant has discovered surprising, distinctive water chemistry behavior and characteristics that occur when DMH is present in the water. This can be leveraged advantageously to provide new methods for managing a body of recreational water. Various embodiments of this nature will now be described.

### Determining DMH Presence

Some embodiments of the present disclosure provide a method for assessing whether a recreational body of water that previously contained 5,5-dimethylhydantoin (DMH) still contains DMH.

The method includes taking one or more oxidation-reduction potential (ORP) measurements for the water while it has a free chlorine level in a range of about 0.5 to 1.0 ppm. When such measurement(s) are taken, the water preferably has a pH in a range of about 7.2-7.8. In some cases, a single ORP measurement is taken. In other cases, a series of ORP measurements is taken. In such cases, the series of ORP measurements can optionally be taken over a period of multiple days. For example, a series of ORP measurements can optionally include three or more ORP measurements taken respectively on three or more days, or even six or more ORP measurements taken respectively on six or more days. When a series of ORP measurements is taken over a period of multiple days, those measurements can optionally be consecutive measurements and/or the multiple days can optionally be consecutive days. In some cases, the series of measurements comprises a plurality of measurements taken over a period of at least 10 days, at least 15 days, or at least 20 days. Reference is made to the non-limiting example of FIG. 1. If desired, multiple measurements can be taken on each of multiple days.

ORP can be measured in any known fashion using conventional equipment for measuring the ORP of a pool, hot tub, or spa. For example, various conventional ORP meters can be used. One non-limiting example is the EcO Start smart water monitor, which is commercially available from iopool USA Inc. (Chicago, Illinois, USA).

In the present embodiment group, the method includes determining DMH to be present in the water in response to one or more ORP measurements being less than 600 mV, such as between 100 and 600 mV, or even less than 500 mV, such as between 300 and 500 mV. Preferably, such ORP measurement(s) is/are taken while the free chlorine level is in a range of about 0.5-1 ppm, such as from 0.5 to 1.0 ppm. Thus, the method may include determining DMH to be present in the water in response to the one or more ORP measurements being in one or more (optionally all) of the foregoing ranges in combination with the free chlorine level being in a range of about 0.5-1 ppm, such as from 0.5 to 1.0 ppm.

In some cases, the method includes determining DMH to be present in the water in response to at least a majority (or at least 75%, or substantially all) of the measurements of a series of ORP measurements (e.g., any type of measurement series noted herein) being less than 600 mV, such as between 100 and 600 mV, or even less than 500 mV, such as between 300 and 500 mV. Preferably, these ORP measurements are taken while the free chlorine level is in a range of about 0.5-1 ppm, such as from 0.5 to 1.0 ppm. Thus, the method may include determining DMH to be present in the water in response to a majority of the measurements in the series being in one or more (optionally all) of the foregoing ranges in combination with the free chlorine level being in a range of about 0.5-1 ppm, such as from 0.5 to 1.0 ppm. In some cases, the series of ORP measurements includes three or more (or five or more) such measurements that are all consecutive measurements falling within one or more (optionally all) of the foregoing mV ranges.

In some cases, none of the ORP measurements of the noted measurement series are greater than 700 mV, or none are greater than 750 mV. Reference is made to the non-limiting example of FIG. 1, which includes the ORP signature for a series of measurements taken during the initial stage of Example 1 (discussed below). Reference is also made to the non-limiting example of FIG. 2, which includes the ORP signature for a series of measurements taken during the initial stage of Example 2. Furthermore, reference is made to the non-limiting example of FIG. 3, which includes the ORP signature for a series of measurements taken during the decline stage of Example 3. Examples 2 and 3 are discussed below.

If desired, the noted series of measurements (showing ORP levels within one or more of the ranges noted above) may span at least a majority (optionally substantially an entirety) of any initial stage and/or the decline stage. This may be the case for any embodiment wherein a series of measurements is taken during any initial stage and/or the decline stage.

In the present embodiments, the one or more ORP measurements preferably are taken during a timeframe when the ORP signature is staying generally constant (e.g., within a range of 300-650 mV, or 250-600 mV, such as 300-600 mV) and/or exhibiting a decline trend. This can optionally be the case for any embodiment of the present group, which involves determining the presence of DMH. As will be appreciated by skilled artisans, there tend to be some variations (when firing the jets of a hot tub as a measurement is being taken, etc.) that can cause the ORP signature not to follow a perfectly horizontal slope or constant downward incline. This can be seen in the non-limiting examples of FIGS. 1 and 2. The ORP signature, however, does show a generally constant level and/or a general (e.g., gradual) decline.

In certain embodiments, in response to the one or more ORP measurements being less than 600 mV, such as between 100 and 600 mV, or even less than 500 mV, such as between 300 and 500 mV, the method may include providing indicia reflecting the measured ORP level(s), notifying an operator that DMH is present in the water, and/or indicating that the water is in a desired (e.g., normal use) stage of operation. This may be done, for example, in cases where the method comprises using a controller, such as may be incorporated into (or operably coupled with) a testing meter configured to take (and/or process data from) such measurements and display such indicia.

In one subgroup of the present embodiment group, the water of the recreational body of water is salt water. Any method of the present disclosure can optionally be carried out with a recreational body of salt water. Thus, the present methods can optionally include adding sodium chloride, such as by adding pool salt, to the recreational body of water. Moreover, in some embodiments of this nature, the method includes operating an electrolytic chlorine generator in the salt water.

In many cases, the recreational body of water is a swimming pool, hot tub, or spa. In such cases, one or more bathers may enter the water and use the swimming pool, hot tub, or spa while the ORP level of the water is less than 600 mV, such as between 100 and 600 mV, or even less than 500 mV, such as between 300 and 500 mV. As noted above, conventional ORP wisdom would consider that recreational water with such low ORP is likely contaminated and should not be used. Applicant has discovered, however, that when the water includes DMH, it can surprisingly be in good condition for bather use (e.g., can have free chlorine in a desired range of about 0.5-1 ppm) at lower ORP levels than previously considered suitable.

In the present methods, the free chlorine level of the water preferably is maintained in the range of about 0.5 to 1.0 ppm for a period of at least 20 days, at least 25 days, or at least 30 days without adding any more DMH to the water during the cycle time. In some cases, prior to that period, the method includes adding DMH to the body of water to establish therein at least 35 ppm DMH, at least 40 ppm DMH, at least 45 ppm DMH, or at least 50 ppm DMH, or more. Moreover, the free chlorine level preferably remains in this range (optionally together with the pH remaining in a range of about 7.2-7.8) during such cycle time. Thus, during an initial stage and/or the decline stage, the free chlorine and/or pH levels preferably remain in one or both of these noted ranges. In other embodiments, though, the method involves adding DMH more frequently.

DMH in a recreational body of water decomposes over time. Thus, the amount of DMH present in such water decreases with time. In some embodiments of the present method, it would be advantageous to assess (at a time later than when the water was determined to contain DMH), whether the recreational body of water has run out of DMH. Preferably, the method is simple, yet reliable and/or at least substantially objective.

Thus, in some of the present embodiments, the method further includes taking a subsequent ORP measurement for the water at a later time, and determining DMH to be absent from the water at the later time in response to the ORP measurement being greater than 600 mV, greater than 650 mV, greater than 700 mV, or greater than 750 mV. In some cases, a series of subsequent ORP measurements is taken, and DMH is determined to be absent from the water in response to at least a majority (or at least 75%, or substantially all) of the subsequent ORP measurements being greater than 600 mV, greater than 650 mV, greater than 700 mV, or greater than 750 mV. In such cases, the series of subsequent ORP measurements can optionally include three or more ORP measurements, or six or more ORP measurements. Additionally or alternatively, the series of subsequent ORP measurements can optionally be taken over a period of multiple days, such as three or more days, or six or more days. In some cases, the series of subsequent ORP measurements includes three or more (or five or more) such measurements that are all consecutive measurements falling within one or more (optionally all) of the foregoing mV ranges. Any type of measurement series disclosed herein can be used for such a series of subsequent ORP measurements.

In embodiments of the present group where one or more subsequent ORP measurements are taken at a time later than when DMH was determined to have been present in the water (i.e., by taking the above-noted DMH measurement(s)), the ORP level preferably is, at some time prior to taking the subsequent ORP measurement(s), in a range of less than 500 mV, such as between 300 and 500 mV.

Furthermore, in embodiments where one or more subsequent ORP measurements are taken, such measurement(s) are, in some cases, taken at a time later than (and/or within 1-3 days from, such as within 1-2 days from) when a peak of free chlorine level occurs in the water. When such a peak occurs, the free chlorine level preferably exceeds 3 ppm, exceeds 4 ppm, or exceeds 5 ppm. Additionally or alternatively, when such a peak of free chlorine occurs, the ORP level preferably exceeds 750 mV. This can optionally be the case for any embodiment wherein one or more subsequent ORP measurements are taken. Reference is made to the non-limiting examples of FIGS. 1-3.

In response to the one or more subsequent ORP measurements being greater than 600 mV, greater than 650 mV, or greater than 700 mV, the method preferably includes adding DMH to the water in an amount sufficient to reduce ORP level. Doing so preferably reduces the ORP level to less than 500 mV, such as between 300 and 500 mV. In some cases, adding DMH to the water in this manner may reduce the ORP level to as low as 100 mV. In certain non-limiting examples, this addition of DMH to the water establishes therein at least 20 ppm DMH, at least 25 ppm DMH, at least 35 ppm DMH, at least 45 ppm, at least 55 ppm DMH, or even more DMH. In some cases, the amount of DMH added to the body of water establishes between about 20 and about 100 ppm DMH in the water, such as between 20 and 90 ppm DMH.

Thus, the method may further include taking a series of subsequent ORP measurements for the water. In some embodiments of this nature, the method comprises, in response to the subsequent measurements reflecting a gradual decrease of ORP level in the water, determining that the DMH present in the water is undergoing decomposition. Preferably, during such gradual decrease of ORP level in the water, the free chlorine level is maintained in the range of about 0.5 to 1.0 ppm, such as from 0.5 to 1.0. Furthermore, the method can advantageously include extrapolating such a gradual decrease of ORP level in the water to predict an exhaustion time when the DMH in the water will be gone. The method may then advantageously include adding more DMH to the water before the exhaustion time.

### Determining DMH Absence

As noted above, DMH in a recreational body of water decomposes over time; the amount of DMH present in the water thus decreases with time. It would be advantageous to provide a method for assessing whether a recreational body of water that previously contained DMH has run out of DMH. Preferably, the method is simple, yet reliable and/or at least substantially objective.

An initial step in such a method comprises taking one or more oxidation-reduction potential (ORP) measurements for the water. When such measurement(s) are taken, the water preferably has a free chlorine level of at least 0.5 ppm and/or a pH in a range of about 7.2-7.8. In some cases, a single ORP measurement is taken. In other cases, a series of ORP measurements is taken. In such cases, the series of ORP measurements can optionally be taken over a period of multiple days. For example, a series of ORP measurements can optionally include three or more ORP measurements taken respectively on three or more days, or even six or more ORP measurements taken respectively on six or more days. When a series of ORP measurements is taken over a period of multiple days, those multiple days can optionally be consecutive days. If desired, multiple measurements can be taken on each of multiple days.

In one group of embodiments, the method includes determining DMH to be absent from the water in response to one or more ORP measurements being greater than 600 mV, greater than 650 mV, greater than 700 mV, or greater than 750 mV. When these one or more ORP measurements are taken, the free chlorine level preferably is at least 0.5 ppm, and may exceed 0.5-1 ppm free chlorine (it may even be greater than 1.5, e.g., greater than 2). This can optionally be the case for any embodiment of the present group. In the non-limiting example of FIG. 1, the ORP signature approaches 800 mV at 60-61 days. In the non-limiting example of FIG. 2, the ORP signature is greater than 750 mV at about day 37. In the non-limiting example of FIG. 3, the ORP signature is greater than 750 mV at about day 18. This reflects that the DMH in the water has run out.

In some cases, the method comprises determining DMH to be absent from the water in response to at least one ORP measurement being greater than 800 mV. When such an ORP measurement is taken, the free chlorine level will typically exceed 0.5-1 ppm free chlorine (e.g., it may be greater than 1.5, e.g., greater than 2).

In certain embodiments of the present group, a series of ORP measurements is taken, and DMH is determined to be absent from the water in response to at least a majority (or at least 75%, or even substantially all) of the ORP measurements being greater than 600 mV, greater than 650 mV, greater than 700 mV, or greater than 750 mV. In some cases, the series of ORP measurements includes three or more (or five or more) such measurements that are all consecutive measurements falling within one or more of the foregoing mV ranges. Any type of measurement series disclosed herein can be used for such a series of ORP measurements.

In some cases, the one or more ORP measurements are taken at least 5 days (or at least 10 days) after DMH was most recently added to the water. This can optionally be the case for any embodiment of the present group, which involves determining DMH to be absent from the body of water.

In one subgroup of the present embodiment group, the water of the recreational body of water is salt water. Thus, the present methods can optionally include adding sodium chloride, such as by adding pool salt, to the recreational body of water. In certain embodiments of the present subgroup, the method includes operating an electrolytic chlorine generator in the salt water. Whether or not the water is salt water, the recreational body of water can optionally be a swimming pool, hot tub, or spa. This can optionally be the case for any embodiment of the present disclosure.

In the present embodiments, at some time prior to taking such ORP measurement(s), the ORP level of the water preferably is in a range of less than 500 mV, such as between 300 and 500 mV. In such cases, the most recent addition of DMH to the water may have occurred before the ORP level was in one or both of these ranges.

Furthermore, in some of the present embodiments, the one or more ORP measurements are taken at a time later than (and/or within 1-3 days of, such as within 1-2 days of) when a peak of the free chlorine in the water occurs. Applicant has discovered that when DMH is added to a recreational body of water, the DMH decomposes over time and eventually runs out. Moreover, Applicant has discovered that when the DMH runs out, there is a peak of the free chlorine level. Reference is made to the non-limiting example of FIG. 1, which reflects a peak of free chlorine level at about 60-61 days. Reference is also made to the non-limiting example of FIG. 2, which reflects a peak of free chlorine level at about day 37. Finally, reference is made to the non-limiting example of FIG. 3, which reflects a peak of free chlorine level at about day 18. When such a peak occurs, the free chlorine level preferably exceeds 3 ppm, exceeds 4 ppm, or exceeds 5 ppm. Additionally or alternatively, when such a peak of free chlorine occurs, the ORP level preferably exceeds 750 mV.

In certain embodiments, in response to the one or more ORP measurements being greater than 600 mV, greater than 650 mV, greater than 700 mV, greater than 750 mV, or greater than 800 mV, the method may include providing indicia reflecting the measured ORP level(s), notifying an operator that DMH is absent from the water, indicating that the water is in a DMH refill stage of operation, and/or prompting an operator to add DMH to the water. This may be done, for example, in cases where the method comprises using a controller, as may be incorporated into (or operably coupled with) a testing meter configured to take (and/or process data from) such measurements and display such indicia.

In response to the one or more ORP measurements being greater than 600 mV, greater than 650 mV, greater than 700 mV, greater than 750 mV, or greater than 800 mV, the method preferably includes adding DMH to the water in an amount sufficient to reduce ORP level. Doing so preferably reduces the ORP level to less than 500 mV, such as between 300 and 500 mV. This also preferably lowers the free chlorine level down to the desired range of about 0.5-1 ppm. In certain non-limiting examples, this addition of DMH to the water establishes therein at least 20 ppm DMH, at least 25 ppm DMH, at least 35 ppm DMH, at least 45 ppm, at least 55 ppm DMH, or even more DMH. In some cases, the amount of DMH added to the body of water establishes between about 20 and about 100 ppm DMH in the water, such as between 20 and 90 ppm DMH.

In many cases, the recreational body of water is a swimming pool, hot tub, or spa. In such cases, after adding DMH to the water, one or more bathers may enter the water and use the swimming pool, hot tub, or spa while the ORP level of the water is less than 500 mV, such as between 300 and 500 mV, or even as low as 100 mV. As noted above, Applicant has surprisingly discovered that when the water includes DMH, it can be in good condition for bather use (e.g., can have free chlorine in a desired range of about 0.5-1 ppm) at lower ORP levels than previously considered suitable.

After adding DMH to the water, the free chlorine level preferably is maintained in the range of about 0.5 to 1.0 ppm for a cycle time of at least 20 days, at least 25 days, or at least 30 days without adding any more DMH to the water during the cycle time. In some embodiments of this nature, when the DMH is added to the body of water, it establishes therein at least 35 ppm DMH, at least 40 ppm DMH, at least 45 ppm DMH, or at least 50 ppm DMH, or more. In other embodiments, though, the method involves adding DMH more frequently.

The present method may further include taking a series of subsequent ORP measurements for the water. In some embodiments of this nature, the method comprises, in response to the subsequent measurements reflecting a gradual decrease of ORP level in the water, determining that the DMH present in the water is undergoing decomposition. Preferably, during such gradual decrease of ORP level in the water, the free chlorine level is maintained in the range of about 0.5 to 1.0 ppm.

Furthermore, the method can advantageously include extrapolating the gradual decrease of ORP level in the water to predict an exhaustion time when the DMH in the water will be gone. The method may then advantageously include adding more DMH to the water before the exhaustion time.

The present disclosure refers to the DMH running out or being absent at various times. As will be appreciated by a person of ordinary skill in this field given the present disclosure as a guide, at those times, there may still be a small residual amount of DMH left in the water. At this point, however, any remaining amount of DMH is small enough that the ORP signature shows a valley at which point the ORP increases dramatically (and along with that steep ORP incline, the free chlorine level increases above the desired range of about 0.5 to 1 ppm). At any point in the present disclosure where reference is made to the DMH running out or being absent, this is to be understood to also disclose that the DMH can be at least substantially entirely gone from the water, i.e., either entirely gone or substantially gone.

### Determining DMH Decomposition

The foregoing embodiments include methods of managing a recreational body of water that is known to have previously contained DMH such that, after time has passed so that at least some of the DMH has decomposed, it is possible to assess whether there is still DMH remaining in the water or whether the DMH has run out. In addition to providing methods that offer those capabilities, it would be advantageous to provide methods that: (i) involve a recreational body of water including DMH, and (ii) assess its behavior in terms of decreasing DMH content.

One embodiment group of the present disclosure provides such a method. The method involves testing a recreational body of water that includes 5,5-dimethylhydantoin (DMH). As an initial step, the method includes taking a series of oxidation-reduction potential (ORP) measurements for the water, preferably while it has a free chlorine level in a range of about 0.5 to 1.0 ppm, such as from 0.5 to 1.0 ppm. The series of measurements may be taken over a period of multiple days (optionally consecutive days), such as three or more days, six or more days, 10 or more days, 12 or more days, or even 14 or more days. Thus, in the first step of the present method, any measurement series noted herein (i.e., any noted number of measurements, taken over any noted period of time) may be used.

As discussed above, the recreational body of water exhibits a decline stage, when the DMH content in the water decreases and there is a corresponding gradual decrease in the ORP level of the water. In some cases, the decline stage may span about 1-2 weeks. Reference is made to the non-limiting, schematic example of Graph 1 above. Also, FIG. 1 shows an example wherein the decline stage spans about two weeks. FIG. 3 shows another example; here, the decline stage spans about 11-15 days. In other cases, the decline stage spans a shorter time period, such as about 2-6 days. Reference is made to FIG. 2, which shows a decline stage spanning about 2-4 days. In the present group of embodiments, it is therefore preferable to take the series of ORP measurements over a period of multiple days.

The method comprises, in response to the ORP measurements reflecting a gradual decrease of ORP level in the water, determining that the DMH present in the water is undergoing decomposition. Preferably, the free chlorine level is maintained in the range of about 0.5 to 1.0 ppm during the gradual decrease of ORP level in the water. This is the case for the decline stages in the non-limiting examples of FIGS. 1-3. Furthermore, during the decline stage (or at least when some of the decline stage ORP measurements are taken), the water preferably has a pH in a range of about 7.2-7.8.

In certain embodiments, in response to the ORP measurements reflecting a gradual decrease of ORP level in the water, the method may include providing indicia reflecting the measured ORP levels, notifying an operator that DMH in the water is decreasing, and/or indicating that the water is in a DMH decline stage of operation. This may be done, for example, in cases where the method comprises using a controller, as may be incorporated into (or operably coupled with) a testing meter configured to take (and/or process data from) such measurements and display such indicia.

In one subgroup of the present embodiment group, the water of the recreational body of water is salt water. Thus, the present methods can optionally include adding sodium chloride, such as by adding pool salt, to the recreational body of water. In some embodiments of the present subgroup, the method includes operating an electrolytic chlorine generator in the salt water. Whether or not the water is salt water, the recreational body of water can optionally be a swimming pool, hot tub, or spa. This can optionally be the case for any embodiment of the present disclosure.

In the present embodiment group, the method can optionally include extrapolating the gradual decrease of ORP level in the water to predict an exhaustion time when the DMH in the water will be gone. This can be done, for example, by graphing or otherwise determining a trend line for the series of measurements so as to predict the exhaustion time. Reference is made to Graph 1 above, notably the illustrated decline stage and the arrow at the end of the graph line encompassed by the dashed-line box. Here, the graph line and the arrow at its end schematically illustrate extrapolating the measured data to predict when DMH in the water will run out.

In embodiments that include extrapolating a gradual decrease of ORP level in the water to predict an exhaustion time when the DMH in the water will be gone, the method may include providing indicia reflecting the predicted exhaustion time, and/or prompting an operator to add DMH within a certain amount of time. This may be done, for example, in cases where the method comprises using a controller, as may be incorporated into (or operably coupled with) a testing meter configured to take (and/or process data from) such measurements and display such indicia.

In managing a recreational body of water that includes DMH, it can be advantageous to be able to predict when the DMH will run out (i.e., the exhaustion time). This can enable, for example, adding more DMH to the water before the exhaustion time. Preferably, more DMH is added before the ORP level increases above 750 mV and/or before the free chlorine level increases above 3 ppm. In certain non-limiting examples, this addition of DMH to the water establishes therein at least 20 ppm DMH, at least 25 ppm DMH, at least 35 ppm DMH, at least 45 ppm, at least 55 ppm DMH, or even more DMH. In some cases, the amount of DMH added to the body of water establishes between about 20 and about 100 ppm DMH in the water, such as between 20 and 90 ppm DMH.

After adding more DMH to the water, the free chlorine level of the water preferably is maintained in the range of about 0.5 to 1.0 ppm for a cycle time of at least 10 days, at least 20 days, at least 25 days, or at least 30 days without adding more DMH to the water during the cycle time.

In many cases, the recreational body of water is a swimming pool, hot tub, or spa. In such cases, before and/or after adding more DMH to the water, one or more bathers may enter the water and use the swimming pool, hot tub, or spa. This may occur while the ORP level of the water is less than 650 mV, such as between 100 and 650 mV, or less than 600 mV, such as between 100 and 600 mV, or even less than 500 mV, such as between 300 and 500 mV. As noted above, conventional ORP wisdom would consider that recreational water with such low ORP is likely contaminated and should not be used. Applicant has discovered, however, that when the water includes DMH, it can surprisingly be in good condition for bather use (e.g., can have free chlorine in a desired range of about 0.5-1 ppm) at lower ORP levels than previously considered suitable.

### Example 1

FIG. 1 is a graph of ORP measurements taken on a hot tub. Example 1 involved a 2017 Bullfrog R7 426-gallon hot tub, using a salt chlorine generator with salt. Initially, the water in the hot tub did not contain any DMH. Thus, the first ORP measurement was taken when there was no DMH in the water, and the free chlorine and ORP were high. For example, the ORP was initially about 800 mV. DMH was then added to the water; the DMH at start was about 80 ppm. This lowered the ORP to about 300-600 mV. There was then an initial stage of relatively constant ORP, with ORP levels oscillating generally around (e.g., with some measurements higher than, and other measurements lower than) 500 mV. During this initial stage, most of the ORP measurements were between 400 and 600 mV. In Example 1, the initial stage lasted about 35 days. As DMH in the water decomposed, the ORP level gradually decreased. During this decline stage, the free chlorine remained substantially constant, e.g., at about 0.5-1 ppm. Once the DMH ran out, the free chlorine peaked at about 61 days. Here, the ORP signature was greater than 750 mV. With continued reference to FIG. 1, the free chlorine at day 60 was about 1 ppm, whereas the free chlorine at day 61 was about 4.5 ppm. In this example, the hot tub was not maintained past 61 days.

### Example 2

FIG. 2 is a graph of ORP measurements taken on another hot tub. Example 2 involved a 2018 Jacuzzi J-585 440-gallon hot tub, using a salt chlorine generator with salt. Initially, the water in the hot tub did not contain DMH or was at least substantially free of DMH (there may have been a small residual amount of DMH from prior testing cycles, at the end of each of which the DMH at least substantially ran out). Thus, the first ORP measurement was taken when there was no DMH in the water (or it was at least substantially free of DMH), so the free chlorine and ORP were high. For example, the ORP was initially about 800 mV. DMH was then added to the water, to provide DMH in the water at about 50 ppm (plus any small residual, as mentioned above). This lowered the ORP to about 300-500 mV. The ORP remained relatively constant during an initial stage, up to about day 28. From about day 28 through about day 31, there was a decline stage. Here, as DMH in the water decomposed, the ORP level decreased. During this decline stage, the free chlorine remained substantially constant, e.g., at about 0.5-1 ppm. Once the DMH ran out, the free chlorine peaked at about 37 days. Here, the ORP signature was greater than 750 mV. In this example, the free chlorine at day 31 was about 0.5 ppm, whereas the free chlorine at day 37 was about 7.6 ppm. In this example, the hot tub was not maintained after the DMH ran out and the ORP peaked.

### Example 3

FIG. 3 is a graph of ORP measurements taken in a prior testing cycle with the same hot tub as in Example 2. In Example 3, prior to the initial ORP measurement, roughly 25 ppm of DMH was added to the water. As DMH in the water decomposed, the ORP level gradually decreased. During this decline stage, the free chlorine remained substantially constant, e.g., at about 0.5-1 ppm. Once the DMH ran out, the free chlorine peaked at about 18 days. Here, the ORP signature was greater than 750 mV. In this example, the free chlorine at day 15 was about 1 ppm, whereas the free chlorine at day 18 was about 9 ppm.

With respect to FIGS. 2 and 3, if additional measurements had been taken between the last two measurement points, then each of these last two graphs would be expected to show the incline up to the illustrated peak being steeper. As can be appreciated by referring to FIG. 1, once the DMH runs out, the ORP signature tends to increase dramatically. This slope up to the peak therefore tends to be quite steep. Also, once the DMH runs out, there tends to be an increase of the free chlorine level, such that after the ORP valley occurs, the free chlorine level increase above the preferred range of about 0.5-1 ppm.

### Startup, Water Conditions

A recreational body of water can be set-up in various ways. In many cases, the body of water is a pool, hot tub, or spa. Preferably, the body of water is provided with one or more chlorinating agents, as well as DMH. A variety of methods for setting-up such a recreation body of water are described in U.S. patent application Ser. No. 18/649,442, entitled “Free Chlorine Maintained Systems,” the contents of which are incorporated herein by reference.

In certain embodiments, the recreational body of water is a seasonal outdoor pool. In such cases, startup may include the following (the noted steps are not necessarily performed sequentially in the order discussed, as will be readily appreciated by a person of ordinary skill in this area):
1. Initially setting the water temperature of the pool at about 85 degrees Fahrenheit, or otherwise setting to the temperature desired (water temperature will subsequently fluctuate due to outside weather conditions);
2. Adjusting the calcium hardness of the pool water to about 200 ppm;
3. Adjusting the alkalinity of the pool water to a range of about 80-120 ppm;
4. Adjusting the pH of the pool water, if needed, to be within a range of about 7.2-7.8;
5. Adding chlorine, such as by adding bleach or calcium hypochlorite. Bring total chlorine level to a range of about 1-20 ppm, perhaps 3-5 ppm;
6. Adding DMH. This can be added in bulk, e.g., solid form. Bring concentration of DMH to a range of about 10-200 ppm, such that the free chlorine is in a range of about 0.5-1.0 ppm;
7. Adjusting the pH of the pool water, if needed, to be within a range of about 7.2-7.8; and
8. Adding algaecide, if desired.

The foregoing base conditions are common for pool set-up conditions and can be established using known pool products to create a recreational body of water that is suitable for bather use. It is to be appreciated, however, that these base conditions may be different for different pools, based on local environmental conditions, bather loads, personal preference, etc. Thus, the foregoing conditions are not limiting.

In other embodiments, the recreational body of water is a hot tub or spa. In such cases, the startup may include the following steps.
1. Fill the container with fresh tap water, with softener partially bypassed to give about 200 parts per million of calcium hardness. Measure and record calcium hardness (Calcium Hardness drop test can be performed, e.g., using a Taylor™ kit K-2006).
2. Adjust the water temperature setting to the temperature desired, such as at least 80 degrees Fahrenheit (if the hot tub or spa is outdoors, its water temperature will fluctuate due to outside weather conditions). Water temperature can be measured, for example, using a Thermoworks™ Superfast Digital Thermometer.
3. Adjust the alkalinity of the water to a range of about 80-120 ppm. This may be done, for example, either by adding sodium bicarbonate (e.g., “Alkanity Up”) to raise the alkalinity, or by adding sodium bisulfate (e.g., “pH Down”) to lower the alkalinity. As one example, 95% sodium bisulfate can be obtained from various commercial suppliers, such as Innovative Water Care, LLC (Alpharetta, Georgia, USA). Alkalinity drop test can be performed, e.g., using a Taylor™ kit K-2006.
4. Adjust the pH of the water, if needed, to be within a range of about 7.2-7.8.
5. Add chlorine, such as by adding bleach or calcium hypochlorite. Bring total chlorine level to a range of about 1-20 ppm, perhaps 3-5 ppm.
6. Add DMH. This can be added in bulk, e.g., solid form. Bring concentration of DMH to a range of about 10-200 ppm, such that the free chlorine is in a range of about 0.5-1.0 ppm. One non-limiting example may include adding about 1.38 ounces of 97% purity DMH to get to about 10 ppm in 1,000 gallons of water.
7. If needed, after 1 day, adjust the pH of the water to be within a range of about 7.2-7.8, e.g., by running the jets until the pH is in this range (the pH level of the water can be measured, for example, using an Oakton™ EcoTestr™ pH 2 meter).
8. If desired, measure total chlorine and, if necessary, adjust so it is at least 3 ppm, such as greater than 5 ppm, or greater than 7 ppm.

In still other embodiments, the water of the recreational body of water comprises (e.g., is) salt water. In some embodiments of this nature, an electrolytic chlorine generator is coupled operatively with the recreational body of water. Furthermore, the water can optionally be salt water. Thus, the present methods may be performed with a pool, hot tub, or spa filled with salt water. In such cases, the startup may include the following steps.
1. Fill the container with fresh tap water, with softener partially bypassed to give about 200 parts per million of calcium hardness. Measure and record calcium hardness (Calcium Hardness drop test can be performed, e.g., using a Taylor™ kit K-2006).
2. Adjust the water temperature setting to the temperature desired, such as at least 80 degrees Fahrenheit (if the hot tub or spa is outdoors, its water temperature will fluctuate due to outside weather conditions). Water temperature can be measured, for example, using a Thermoworks™ Superfast Digital Thermometer.
3. Adjust the alkalinity of the water to a range of about 80-120 ppm. This may be done, for example, either by adding sodium bicarbonate (e.g., “Alkanity Up”) to raise the alkalinity, or by adding sodium bisulfate (e.g., “pH Down”) to lower the alkalinity. As one example, 95% sodium bisulfate can be obtained from various commercial suppliers, such as Innovative Water Care, LLC (Alpharetta, Georgia, USA). Alkalinity drop test can be performed, e.g., using a Taylor™ kit K-2006.
4. Adjust the pH of the water, if needed, to be within a range of about 7.2-7.8.
5. Add chlorine, such as by adding bleach or calcium hypochlorite. Bring total chlorine level to a range of about 1-20 ppm, perhaps 3-5 ppm.
6. Add sodium chloride, such as adding pool salt to achieve a level of about 1000-2000 ppm. One non-limiting example may include adding about 8.35 pounds of 99% purity sodium chloride to get to about 1,000 ppm in 1,000 gallons of water. The salt concentration can be measured with Leslie's Salt Test Strips for example (the salt concentration technically depends on the generator design and temperature of the water, 1000-2000 ppm is the most common range for hot tubs, nominal is roughly 1500 ppm).
7. Add DMH. This can be added in bulk, e.g., solid form. Bring concentration of DMH to a range of about 10-200 ppm, such that the free chlorine is in a range of about 0.5-1.0 ppm. One non-limiting example may include adding about 1.38 ounces of 97% purity DMH to get to about 10 ppm in 1,000 gallons of water.
8. Provide a salt chlorine generator and set the operation in accordance with the manufacturer's recommendation to produce a total chlorine level of at least 3 ppm, such as greater than 5 ppm, or greater than 7 ppm. One non-limiting example may be to use the Briidea Salt Chlorine Generator for Hot Tubs & Swim Spas.
9. If needed, after 1 day, adjust the pH of the water to be within a range of about 7.2-7.8, e.g., by running the jets until the pH is in this range (the pH level of the water can be measured, for example, using an Oakton™ EcoTestr™ pH 2 meter).
10. If desired, measure total chlorine and, if necessary, adjust the run time on the salt chlorine generator so that the chlorine level is at least 3 ppm, such as greater than 5 ppm, or greater than 7 ppm.

One non-limiting example of a suitable spa is the 400-gallon Hot Spring® Vanguard® brand spa, which is commercially available from Watkins Wellness (Vista, California, USA). Another suitable spa is the 300-gallon Marquis® Destiny® spa, which is commercially available from Marquis Corp. (Independence, Oregon, USA). Still another suitable hot tub is the Jacuzzi® 440-gallon J-475™ hot tub, which is commercially available from Jacuzzi, Inc. (Irvine, California, USA). Yet another suitable hot tub is the Arctic Spas® 360-gallon Summit spa, which is commercially available from Blue Falls Manufacturing Limited (Thorsby, Alberta, Canada). Still another suitable hot tub is the CalSpas® CS-PL-893B hot tub, which is commercially available from Cal Spas (Pomona, California, USA). Yet another suitable hot tub is the Bullfrog Spas® 453-gallon M7 hot tub, which is commercially available from Bullfrog International LLC (Salt Lake City, Utah, USA).

Chlorine can be added to the water in several different ways, including but not limited to, chlorinating agents comprising liquid bleach (NaClO) or calcium hypochlorite (Ca(ClO)). In some cases, the chlorinating agent comprises or results from liquid bleach, calcium hypochlorite, chlorine gas, lithium hypochlorite, or a combination thereof. If desired, an electrolytic cell can be provided to generate chlorine, e.g., when using salt water.

Once the initial chlorine conditions are established, a maintenance amount of chlorine can be continually or intermittingly added to the body of water through a dispenser, or the like, to maintain the body of water in the desired condition for human recreational use. Typically, the rate of release of chlorine is manually selected, e.g., in the case of a pool, on either an inline dispenser or an in-water dispenser to maintain a free chlorine base (e.g., 0.5-1.0 ppm free chlorine) in the body of water sufficient to maintain the pool in desirable condition for use by bathers. Or, when using salt water, a chlorine generator can be used to provide the maintenance amount of chlorine.

In some embodiments, an operator adjusts a chlorine dispenser to deliver a constant rate of chlorine into the body of water. In such cases, delivering a constant rate of chlorine may be intended to balance the ongoing needs of the body of water as it responds external environment and is considered a base level to handle the typical ongoing chlorine needs. Similarly, when using salt water, a chlorine generator can be operated to provide a constant amount of chlorine.

During use of the recreational body of water, an operator may periodically test the level of chlorine and adjust the rate of chlorine release, e.g., for pool embodiments, from a dispenser in the event the ongoing chlorine needs of a pool changes. The ongoing chlorine needs of a pool, for example, may change due to sunlight or organic debris that may enter the pool, such as but not limited to after a storm. These types of changes in chlorine needs are considered long-term changes and are compensated typically through a manual adjustment of the amount of chlorine delivered to the pool. Or, when using salt water, a chlorine generator can be operated at different levels at different times to change the amount of chlorine as desired to meet changing needs.

In some examples, DMH is added to the body of water followed by the addition of a chlorinating agent until the free chlorine reaches an equilibrium state at about 1 ppm. In another example, chlorine is added to the body of recreational water followed by the addition of sufficient DMH to bring the free chlorine into an equilibrium state, which is about 1 ppm. In still another example, the DMH and chlorine agent may be simultaneously delivered to the body of water either in bulk form or through a container that is thrown or placed in the body of water, for example, a container that dissolves in the body of water to release the chlorinating agent and DMH. Thus, chlorinating agent may initially be added to the body of water before and/or after initially adding DMH to the body of water. Furthermore, when using salt water, a chlorine generator can be operated to provide a desired base level of chlorine.

In some cases, a temperature of the body of water is maintained in a range of about 70° F. to about 104° F., while the alkalinity of the body of water is maintained in a range of between about 80 ppm and about 120 ppm.

In some cases, the method is devoid of adding cyanuric acid (CYA) to the body of water. This can optionally be the case for any embodiment of the present disclosure. In some embodiments of this nature, the body of water is devoid of CYA during any method of this disclosure. This can optionally be the case for any embodiment of the present disclosure. In other embodiments, though, some cyanuric acid is present in the body of water.

Various embodiments have been described. These and other embodiments are within the scope of the following claims.

### EMBODIMENTS

### Group 1

1. A method for assessing whether a recreational body of water that previously contained 5,5-dimethylhydantoin (DMH) still contains DMH, the method comprising:
   taking a series of oxidation-reduction potential (ORP) measurements for the water while it has a free chlorine level in a range of about 0.5 to 1.0 ppm, and
   determining DMH to be present in the water in response to at least a majority of the ORP measurements being in a range of between 100 and 600 mV.
2. The method of embodiment 1 wherein the water has a pH in a range of about 7.2-7.8.
3. The method of embodiment 1 or 2 wherein the water is salt water.
4. The method of any one of the preceding embodiments further comprising operating an electrolytic chlorine generator in the salt water.
5. The method of any one of the preceding embodiments wherein the series of ORP measurements is taken over a period of multiple days.
6. The method of any one of the preceding embodiments wherein the series of ORP measurements includes three or more ORP measurements taken respectively on three or more days.
7. The method of any one of the preceding embodiments wherein the series of ORP measurements includes six or more ORP measurements taken respectively on six or more days.
8. The method of any one of the preceding embodiments wherein at least one of the ORP measurements is in a range of between 300 and 500 mV.
9. The method of any one of the preceding embodiments wherein none of the ORP measurements are greater than 700 mV.
10. The method of any one of the preceding embodiments wherein the recreational body of water is a swimming pool, hot tub, or spa.
11. The method of embodiment 10 wherein one or more bathers enter the water and use the swimming pool, hot tub, or spa while ORP level of the water is in the range of between 100 and 600 mV.
12. The method of embodiment 10 or 11 wherein one or more bathers enter the water and use the swimming pool, hot tub, or spa while ORP level of the water is in a range of between 300 and 500 mV.
13. The method of any one of the preceding embodiments wherein the free chlorine level of the water is maintained in the range of about 0.5 to 1.0 ppm for a cycle time of at least 20 days without adding any more DMH to the water during the cycle time.
14. The method of any one of the preceding embodiments further comprising taking a series of subsequent ORP measurements for the water at a later time, and determining DMH to be absent from the water at the later time in response to at least a majority of the subsequent ORP measurements being greater than 600 mV.
15. The method of any one of the preceding embodiments wherein the series of subsequent ORP measurements includes three or more ORP measurements.
16. The method of any one of the preceding embodiments wherein the series of subsequent ORP measurements is taken over a period of multiple days.
17. The method of any one of the preceding embodiments wherein the subsequent ORP measurements are taken at a time later than when a peak of the free chlorine level occurs.
18. The method of embodiment 17 wherein, when the peak of the free chlorine level occurs, the free chlorine level exceeds 3 ppm.
19. The method of embodiment 17 or 18 wherein, when the peak of the free chlorine level occurs, ORP level in the water exceeds 750 mV.
20. The method of any one of the preceding embodiments further comprising taking a subsequent ORP measurement for the water at a later time, and determining DMH to be absent from the water at the later time in response to the subsequent ORP measurement being greater than 700 mV.
21. The method of any one of the preceding embodiments wherein the subsequent ORP measurement is taken at a time later than when a peak of the free chlorine level occurs.
22. The method of any one of the preceding embodiments wherein, when the peak of the free chlorine level occurs, the free chlorine level exceeds 3 ppm.
23. The method of any one of the preceding embodiments wherein, when the peak of the free chlorine level occurs, ORP level in the water exceeds 750 mV.
24. The method of any one of the preceding embodiments further comprising taking a series of subsequent ORP measurements for the water at a later time and, in response to at least a majority of the subsequent ORP measurements being greater than 600 mV, adding DMH to the water in an amount sufficient to reduce ORP level.
25. The method of any one of the preceding embodiments wherein, at some time prior to taking the series of subsequent ORP measurements, ORP level of the water is in a range of between 300 and 500 mV.
26. The method of embodiment 24 or 25 wherein said adding DMH to the water reduces ORP level to the range of between 300 and 500 mV.
27. The method of any one of the preceding embodiments wherein said adding DMH to the water reduces ORP level to as low as 100 mV.
28. The method of any one of the preceding embodiments further comprising taking a series of subsequent ORP measurements for the water and, in response to said subsequent ORP measurements reflecting a gradual decrease of ORP level in the water, determining that the DMH present in the water is undergoing decomposition.
29. The method of embodiment 28 wherein the free chlorine level is maintained in the range of about 0.5 to 1.0 ppm during the gradual decrease of ORP level in the water.
30. The method of embodiment 28 or 29 further comprising extrapolating the gradual decrease of ORP level in the water to predict an exhaustion time when the DMH in the water will be gone.
31. The method of embodiment 30 further comprising adding more DMH to the water before the exhaustion time.
32. A method for assessing whether a recreational body of water that previously contained 5,5-dimethylhydantoin (DMH) still contains DMH, the method comprising:
   taking an oxidation-reduction potential (ORP) measurement for the water while it has a free chlorine level in a range of about 0.5 to 1.0 ppm, and
   determining DMH to be present in the water in response to the ORP measurement being below 500 mV.
33. The method of any one of the preceding embodiments wherein the water has a pH in a range of about 7.2-7.8.
34. The method of any one of the preceding embodiments wherein the water is salt water.
35. The method of any one of the preceding embodiments further comprising operating an electrolytic chlorine generator in the salt water.
36. The method of any one of the preceding embodiments wherein the recreational body of water is a swimming pool, hot tub, or spa.
37. The method of any one of the preceding embodiments wherein one or more bathers enter the water and use the swimming pool, hot tub, or spa while ORP level of the water is below 500 mV.
38. The method of any one of the preceding embodiments wherein the free chlorine level of the water is maintained in the range of about 0.5 to 1.0 ppm for a cycle time of at least 20 days without adding more DMH to the water during the cycle time.
39. The method of any one of the preceding embodiments further comprising taking a series of subsequent ORP measurements for the water at a later time, and determining DMH to be absent from the water at the later time in response to at least a majority of the subsequent ORP measurements being greater than 600 mV.
40. The method of any one of the preceding embodiments wherein the series of subsequent ORP measurements includes three or more ORP measurements.
41. The method of any one of the preceding embodiments wherein the series of subsequent ORP measurements is taken over a period of multiple days.
42. The method of any one of the preceding embodiments wherein the subsequent ORP measurements are taken at a time later than when a peak of the free chlorine level occurs.
43. The method of any one of the preceding embodiments wherein, when the peak of the free chlorine level occurs, the free chlorine level exceeds 5 ppm.
44. The method of any one of the preceding embodiments wherein, when the peak of the free chlorine level occurs, ORP level in the water exceeds 750 mV.
45. The method of any one of the preceding embodiments further comprising taking a subsequent ORP measurement for the water at a later time, and determining DMH to be absent from the water at the later time in response to the subsequent ORP measurement being greater than 700 mV.
46. The method of any one of the preceding embodiments wherein the subsequent ORP measurement is taken at a time later than when a peak of the free chlorine level occurs.
47. The method of any one of the preceding embodiments wherein, when the peak of the free chlorine level occurs, the free chlorine level exceeds 5 ppm.
48. The method of any one of the preceding embodiments wherein, when the peak of the free chlorine level occurs, ORP level in the water exceeds 750 mV.
49. The method of any one of the preceding embodiments further comprising taking a series of subsequent ORP measurements for the water at a later time and, in response to at least a majority of the subsequent ORP measurements being greater than 600 mV, adding DMH to the water in an amount sufficient to reduce ORP level.
50. The method of any one of the preceding embodiments wherein, at some time prior to taking the series of subsequent ORP measurements, ORP level of the water is a range of less than 500 mV.
51. The method of any one of the preceding embodiments wherein said adding DMH to the water reduces ORP level to the range of less than 500 mV.
52. The method of any one of the preceding embodiments wherein said adding DMH to the water reduces ORP level to as low as 100 mV.
53. The method of any one of the preceding embodiments further comprising taking a series of subsequent ORP measurements for the water and, in response to said subsequent ORP measurements reflecting a gradual decrease of ORP level in the water, determining that the DMH present in the water is undergoing decomposition.
54. The method of any one of the preceding embodiments wherein the free chlorine level is maintained in the range of about 0.5 to 1.0 ppm during the gradual decrease of ORP level in the water.
55. The method of any one of the preceding embodiments further comprising extrapolating the gradual decrease of ORP level in the water to predict an exhaustion time when the DMH in the water will be gone.
56. The method of any one of the preceding embodiments further comprising adding more DMH to the water before the exhaustion time.

### Group 2

1. A method of testing a recreational body of water that includes 5,5-dimethylhydantoin (DMH), the method comprising:
   taking a series of oxidation-reduction potential (ORP) measurements for the water while it has a free chlorine level in a range of about 0.5 to 1.0 ppm, and
   in response to the ORP measurements reflecting a gradual decrease of ORP level in the water, determining that the DMH present in the water is undergoing decomposition.
2. The method of embodiment 1 wherein the water has a pH in a range of about 7.2-7.8.
3. The method of embodiment 1 or 2 wherein the water is salt water.
4. The method of any one of the preceding embodiments further comprising operating an electrolytic chlorine generator in the salt water.
5. The method of any one of the preceding embodiments wherein the free chlorine level is maintained in the range of about 0.5 to 1.0 ppm during the gradual decrease of ORP level in the water.
6. The method of any one of the preceding embodiments further comprising extrapolating the gradual decrease of ORP level in the water to predict an exhaustion time when the DMH in the water will be gone.
7. The method of embodiment 6 further comprising adding more DMH to the water before the exhaustion time.
8. The method of embodiment 7 wherein, after said adding more DMH to the water, the free chlorine level of the water is maintained in the range of about 0.5 to 1.0 ppm for a cycle time of at least 20 days without adding more DMH to the water during the cycle time.
9. The method of any one of the preceding embodiments wherein the recreational body of water is a swimming pool, hot tub, or spa.
10. The method of embodiment 9 wherein, after said adding DMH to the water, one or more bathers enter the water and use the swimming pool, hot tub, or spa while ORP level of the water is in a range of between 100 and 650 mV.
11. The method of embodiment 9 or 10 wherein, after said adding DMH to the water, one or more bathers enter the water and use the swimming pool, hot tub, or spa while ORP level of the water is in a range of between 300 and 500 mV.
12. A method for assessing whether a recreational body of water that previously contained 5,5-dimethylhydantoin (DMH) still contains DMH, the method comprising:
   taking an oxidation-reduction potential (ORP) measurement for the water while it has a free chlorine level of at least 0.5 ppm, and
   determining DMH to be absent from the water in response to the ORP measurement being greater than 700 mV.
13. The method of embodiment 12 wherein said taking the ORP measurement is part of taking a series of ORP measurements for the water, and wherein at least a majority of the series of ORP measurements are greater than 700 mV.
14. The method of embodiment 12 or 13 wherein the series of ORP measurements is taken over a period of multiple days.
15. The method of any one of the preceding embodiments wherein the series of ORP measurements includes three or more ORP measurements taken respectively on three or more days.
16. The method of any one of the preceding embodiments wherein the water has a pH in a range of about 7.2-7.8.
17. The method of any one of the preceding embodiments wherein the water is salt water.
18. The method of any one of the preceding embodiments further comprising operating an electrolytic chlorine generator in the salt water.
19. The method of any one of the preceding embodiments wherein the ORP measurement is taken at a time later than when a peak of the free chlorine level occurs.
20. The method of embodiment 19wherein, when the peak of the free chlorine level occurs, the free chlorine level exceeds 3 ppm.
21. The method of embodiment 19 or 20 wherein, when the peak of the free chlorine level occurs, ORP level in the water exceeds 750 mV.
22. The method of any one of the preceding embodiments further comprising, in response to the ORP measurement being greater than 700 mV, adding DMH to the water in an amount sufficient to reduce ORP level.
23. The method of any one of the preceding embodiments wherein said adding DMH to the water reduces ORP level to below 500 mV.
24. The method of any one of the preceding embodiments wherein said adding DMH to the water reduces ORP level to as low as 100 mV.
25. The method of any one of the preceding embodiments wherein the recreational body of water is a swimming pool, hot tub, or spa.
26. The method of any one of the preceding embodiments wherein, after said adding DMH to the water, one or more bathers enter the water and use the swimming pool, hot tub, or spa while ORP level of the water is in a range of between 100 and 650 mV.
27. The method of any one of the preceding embodiments wherein, after said adding DMH to the water, one or more bathers enter the water and use the swimming pool, hot tub, or spa while ORP level of the water is in a range of between 300 and 500 mV.
28. The method of any one of the preceding embodiments wherein, after said adding DMH to the water, the free chlorine level of the water is maintained in the range of about 0.5 to 1.0 ppm for a cycle time of at least 20 days without adding more DMH to the water during the cycle time.
29. The method of any one of the preceding embodiments further comprising taking a series of subsequent ORP measurements for the water and, in response to said subsequent ORP measurements reflecting a gradual decrease of ORP level in the water, determining that the DMH present in the water is undergoing decomposition.
30. The method of any one of the preceding embodiments wherein the free chlorine level is maintained in the range of about 0.5 to 1.0 ppm during the gradual decrease of ORP level in the water.
31. The method of any one of the preceding embodiments further comprising extrapolating the gradual decrease of ORP level in the water to predict an exhaustion time when the DMH in the water will be gone.
32. The method of any one of the preceding embodiments further comprising adding more DMH to the water before the exhaustion time.

What is claimed is:

1. A method for assessing whether a recreational body of water that previously contained 5,5-dimethylhydantoin (DMH) still contains DMH, the method comprising:
taking a series of oxidation-reduction potential (ORP) measurements for the water while it has a free chlorine level in a range of about 0.5 to 1.0 ppm, and
determining DMH to be present in the water in response to at least a majority of the ORP measurements being in a range of between 100 and 600 mV.

2. The method of claim 1 wherein the water has a pH in a range of about 7.2-7.8.

3. The method of claim 1 wherein the water is salt water.

4. The method of claim 3 further comprising operating an electrolytic chlorine generator in the salt water.

5. The method of claim 1 wherein the series of ORP measurements is taken over a period of multiple days.

6. The method of claim 5 wherein the series of ORP measurements includes three or more ORP measurements taken respectively on three or more days.

7. The method of claim 5 wherein the series of ORP measurements includes six or more ORP measurements taken respectively on six or more days.

8. The method of claim 1 wherein at least one of the ORP measurements is in a range of between 300 and 500 mV.

9. The method of claim 1 wherein none of the ORP measurements are greater than 700 mV.

10. The method of claim 1 wherein the recreational body of water is a swimming pool, hot tub, or spa.

11. The method of claim 10 wherein one or more bathers enter the water and use the swimming pool, hot tub, or spa while ORP level of the water is in the range of between 100 and 600 mV.

12. The method of claim 10 wherein one or more bathers enter the water and use the swimming pool, hot tub, or spa while ORP level of the water is in a range of between 300 and 500 mV.

13. The method of claim 1 wherein the free chlorine level of the water is maintained in the range of about 0.5 to 1.0 ppm for a cycle time of at least 20 days without adding any more DMH to the water during the cycle time.

14. The method of claim 1 further comprising taking a series of subsequent ORP measurements for the water at a later time, and determining DMH to be absent from the water at the later time in response to at least a majority of the subsequent ORP measurements being greater than 600 mV.

15. The method of claim 14 wherein the series of subsequent ORP measurements includes three or more ORP measurements.

16. The method of claim 14 wherein the series of subsequent ORP measurements is taken over a period of multiple days.

17. The method of claim 14 wherein the subsequent ORP measurements are taken at a time later than when a peak of the free chlorine level occurs.

18. The method of claim 17 wherein, when the peak of the free chlorine level occurs, the free chlorine level exceeds 3 ppm.

19. The method of claim 18 wherein, when the peak of the free chlorine level occurs, ORP level in the water exceeds 750 mV.

20. The method of claim 1 further comprising taking a subsequent ORP measurement for the water at a later time, and determining DMH to be absent from the water at the later time in response to the subsequent ORP measurement being greater than 700 mV.

21. The method of claim 20 wherein the subsequent ORP measurement is taken at a time later than when a peak of the free chlorine level occurs.

22. The method of claim 21 wherein, when the peak of the free chlorine level occurs, the free chlorine level exceeds 3 ppm.

23. The method of claim 21 wherein, when the peak of the free chlorine level occurs, ORP level in the water exceeds 750 mV.

24. The method of claim 1 further comprising taking a series of subsequent ORP measurements for the water at a later time and, in response to at least a majority of the subsequent ORP measurements being greater than 600 mV, adding DMH to the water in an amount sufficient to reduce ORP level.

25. The method of claim 24 wherein, at some time prior to taking the series of subsequent ORP measurements, ORP level of the water is in a range of between 300 and 500 mV.

26. The method of claim 25 wherein said adding DMH to the water reduces ORP level to the range of between 300 and 500 mV.

27. The method of claim 24 wherein said adding DMH to the water reduces ORP level to as low as 100 mV.

28. The method of claim 1 further comprising taking a series of subsequent ORP measurements for the water and, in response to said subsequent ORP measurements reflecting a gradual decrease of ORP level in the water, determining that the DMH present in the water is undergoing decomposition.

29. The method of claim 28 wherein the free chlorine level is maintained in the range of about 0.5 to 1.0 ppm during the gradual decrease of ORP level in the water.

30. The method of claim 28 further comprising extrapolating the gradual decrease of ORP level in the water to predict an exhaustion time when the DMH in the water will be gone.

31. The method of claim 30 further comprising adding more DMH to the water before the exhaustion time.
